(19) United States
(12) Patent Application Publication
(10) Pub. No.: US 2025/0281384 A1
(43) Pub. Date: Sep. 11, 2025

(54) POLYESTER, VISCOUS MIXTURE, PROCESS FOR PREPARING SAME AND COSMETIC COMPOSITION

(71) Applicant: BIOSYNTHIS, Saint Cyr Sous Dourdan (FR)

(72) Inventor: Thierry BERNOUD, Saint Cyr Sous Dourdan (FR)

(73) Assignee: BIOSYNTHIS, Saint Cyr Sous Dourdan (FR)

(21) Appl. No.: 18/862,749

(22) PCT Filed: May 2, 2023
(86) PCT No.: PCT/EP2023/061586

§ 371 Date: Jan. 16, 2025

(30) Foreign Application Priority Data
May 2, 2022 (FR) FR2204166

(51) Int. Cl. A61K 8/85; A61K 8/31; A61Q 1/10; A61Q 5/00; C08G 63/16; C08G 63/85

(52) U.S. Cl. CPC A61K 8/85; A61K 8/31; A61Q 1/10; A61Q 5/00; C08G 63/16; C08G 63/85; A61K 2800/591; A61K 2800/95

(57) ABSTRACT

A mixture including: a) at least one polyester resulting from a reaction between at least one dicarboxylic acid dimer and at least one alcohol bearing at least two hydroxyl functions, the at least one polyester having a viscosity of at least 200 000 cSt at 40° C. and a hydroxyl number of less than 30 mg KOH/g, b) at least one biobased hydrocarbon compound. Also, the process obtains the polyester and the polyester. Also, a cosmetic composition includes the mixture and at least one compound chosen from non-volatile oils, volatile oils, glossy oils, pasty fatty substances, gelling agents, film-forming polymers, waxes, antioxidants, pigments, dyes, surfactants and an aqueous phase, and mixtures thereof.

The present application relates to a viscous mixture comprising at least one polyester and at least one biosourced hydrocarbon compound.

The present application also relates to a process for preparing such a mixture.

It also concerns polyester and its preparation process.

Furthermore, the invention relates to the polyester compound and the cosmetic composition comprising the mixture according to the invention.

As part of this request, a number of definitions must be given.

An “alkane” is a saturated hydrocarbon consisting only of carbon and hydrogen atoms linked together by simple covalent bonds whose general formula is CₙH₂ₙ₊₂.

A “straight alkane” is an alkane in which each carbon atom is bonded to a maximum of two carbon atoms.

A “branched alkane” is an alkane in which some carbon atoms are linked to three or even four carbon atoms.

A “Cₓ alkane” is an alkane consisting of x carbon atoms, and the alkane may be linear or branched. For example, dodecane is a C₁₂ alkane.

A “C>x alkane” is an alkane consisting of at least x+1 carbon atoms, and the alkane may be linear or branched. For example, dodecane is a C>10 linear alkane or C12 alkane.

A “C≥x alkane” is an alkane consisting of at least x carbon atoms, said alkane being able to be linear or branched. For example, 2,3,6-trimethylheptane (CAS 4032-93-3) is a C≥10 alkane.

A “Cₓ-C_{y}” alkane is an alkane consisting of x to y carbon atoms, and the alkane may be linear or branched. For example, C₁-C₂ alkanes are methane and ethane.

An “isoalkane” is a branched alkane containing at least one carbon atom linked to at least three carbon atoms including a methyl group.

A “polyester” is a polymer whose main chain consists of an alternating sequence of units derived from at least one dicarboxylic acid and units derived from at least one alcohol comprising at least two hydroxyl functions, these units being linked together by ester functions.

A “hydrocarbon compound” is a compound consisting essentially of carbon and hydrogen atoms.

The “hydroxyl index” is the quantity of free hydroxyls measured according to the NF ISO 4629 4629-2 method. The hydroxyl index is expressed in mg KOH/g.

For the purposes of the present invention, the term “viscosity” refers to the kinematic viscosity which is the quotient of the dynamic viscosity by the density or density of the fluid. The kinematic viscosity is commonly expressed in centistokes (cSt) or mm²·s^{-1.}

The term “mass percentage” refers to the ratio of the mass of a first compound to the total mass of a mixture of compounds or composition, reduced to a percentage. For example, if 10 grams of a compound y are present in a mixture z having a total mass of 100 grams, then the mass percentage of y in z is 10%.

The polyesters according to the invention are characterized by their molecular mass, this is measured by the method known as Size Exclusion Chromatography (SEC) and it is expressed in g/mol.

The polyesters according to the invention are characterized by their colorimetry, this is determined by the APHA-HAZEN method according to the ASTM D 1209 standard and it is expressed in APHA.

A “biosourced” compound or organic composition is defined as one in which the organic carbon present in the compound or composition is 100% of plant origin (¹⁴C) by radiocarbon analysis according to one of the following standards: ASTM D6866, EN 16640 or EN 16785-1.

The “naturalness index” of an organic composition is the percentage of organic carbon of plant origin (¹⁴C) determined by radiocarbon analysis according to one of the following standards: ASTM D6866, EN 16640 or EN 16785-1.

In cosmetic or dermatological products, fine control of the product structure or viscosity is necessary in relation to the intended application and represents one of the critical elements in the user's appreciation of the product. For example, a lipstick, foundation or eye shadow must not flow once applied to the skin, while a hair product such as a gel or serum, for example, must be able to be easily spread by the user on the hair.

Ingredients for adjusting the structural, thickening and gelling characteristics of a cosmetic product include waxes, mineral fillers based on silica in particular, hydrophobized clays such as bentonites, metallic salts of fatty acids, fatty acid esters and sugar or oligosaccharide derivatives functionalized by fatty acids such as dextran palmitate or sucrose esters.

The available structuring agents have disadvantages. Waxes, for example, can leave a greasy feeling on the skin and lead to a matte appearance, a loss of shine.

Structuring products must therefore allow a fine adjustment of the consistency of the product without negatively impacting the feel, durability of the product, coloring and shine in particular. There is therefore a need to offer structuring products that are “spectators” of the other characteristics of the product or, even better, that can improve them. Such products are of great interest because they can reduce the number of ingredients used in a cosmetic composition.

Polyesters derived from the condensation of naturally occurring fatty carboxylic acids and polyols have been considered in recent years as being able to adjust the consistency of cosmetic products while preserving or even improving properties such as shine.

NIPPON FINE CHEMICAL offers oligomers of esters derived from dilinoleic acid dimers and particular dilinoleic diol dimers under the trade names LUSPLAN DD-DA5® and LUSPLAN DD-DA7®. These oligoesters obtained by polycondensation in a heptane solvent have a yellowish colour and cannot replace a thickening or gelling agent due to their low viscosity. In addition, the hydroxyl number of the commercial product LUSPLAN DD-DA7® is between 50 and 55 mg KOH/g, which is therefore higher than the hydroxyl numbers of the polyesters according to the invention.

L'OREAL has filed two patent applications, WO2013191302 and WO2018119762, in which cosmetic compositions containing polyesters with high hydroxyl indices, of the PLANDOOL G type, are disclosed. These polyesters are obtained by the condensation of acid dimers and alcohol dimers, in the presence of monoalcohols such as behenyl alcohol and/or phytosterol derivatives. The presence of these reagents makes it possible to convert some of the free hydroxyl groups resulting from the condensation reaction.

The same type of oligomers is disclosed in patent JP2018123117, in the name of the company NIPPON FINE CHEMICAL. These polyesters are obtained by the condensation between an acid dimer and a diol dimer at high temperature and at atmospheric pressure. The products thus obtained are subsequently diluted to 50 w/w % in a mixture of liquid paraffin and ethylhexyl palmitate, in a proportion of 1:1. On the other hand, the experimental conditions used in this patent application do not make it possible to obtain final polyesters comparable to those of the present invention because their polymerization rate will be lower and therefore their hydroxyl index will be higher due to a greater number of functional groups that have not reacted, and therefore left free.

Polyesters derived in particular from fatty acids have also been proposed by the company CRODA in patent applications EP1962790A1 and EP2004137A1. These polyesters are obtained by condensation of 3 reagents: a dicarboxylic acid, a polyol carrying 3 to 8 hydroxyl functions and a C₁₆-C₃₀ monocarboxylic fatty acid.

The applicant has also proposed polyesters obtained by condensation between dilinoleic acid and 1,4-butanediol, polyesters available under the trade name Viscoplast 14436H. These low viscosity liquid polyesters have in particular been combined with a fatty phase in patent application FR2931670A1 of the company L'OREAL.

The company NITTO DENKO CORPORATION in application EP2853573 presents the manufacture of polyesters used, after crosslinking with isocyanates, in adhesive compositions to be applied to the skin. These polyesters are obtained by condensation between an acid dimer and an alcohol dimer. These condensations are carried out either at very low pressure and without elimination of water, and preferably in two stages, or at atmospheric pressure but with distillation of the water generated during the reaction. The first stage is carried out at a temperature between 170 and 190° C. under a vacuum between 7 and 13 mbar and the second stage at an even lower pressure, between 2 and 8 mbar, and at a temperature between 215 and 235° C. Unlike the polyesters according to the invention, it is necessary for them to have free hydroxyl functions to allow them to be crosslinked with isocyanate-type derivatives.

Due to their biodegradable nature and potential structuring agents, polyesters obtained by condensation of fatty acids and fatty alcohols are of definite interest for the preparation of cosmetic compositions.

Their use has so far been limited due to their low viscosity, hence the use of additional lipophilic thickening or gelling agents, in particular non-biodegradable synthetic polymers such as polyorganosiloxanes copolymers of the polystyrene/copoly(ethylene-propylene) or polystyrene/copoly(ethylene-butylene) type.

The multiplication of ingredients complicates the work of the formulator and requires extremely precise dosage of the latter to obtain properties of interest to the user and also to ensure the stability of these properties over time.

Surprisingly, it was shown that high viscosity polyesters with a low hydroxyl index could be obtained by a condensation reaction in the presence of at least one biosourced hydrocarbon compound.

The mixture according to the invention comprising at least one polyester and at least one biosourced hydrocarbon compound has a viscosity such that it makes it possible to limit, or even eliminate, the use of thickening or gelling agents in cosmetic compositions.

Furthermore, the method of preparing the mixture according to the invention allows the preparation of cosmetic compositions which can have a naturalness index greater than or equal to 90%, more advantageously greater than or equal to 95%.

The invention relates to a mixture comprising:
(a) at least one polyester resulting from a reaction between at least one dicarboxylic acid dimer and at least one alcohol bearing at least two hydroxyl functions, the at least one polyester having a viscosity of at least 200,000 cSt at 40° C. and a hydroxyl index of less than 30 mg KOH/g.
(b) at least one biosourced hydrocarbon compound.

In one embodiment, the polyester has a viscosity of at least 500,000 cSt at 40° C. and a hydroxyl number of less than 30 mg KOH/g.

In one embodiment, the polyester has a viscosity of at least 1,000,000 cSt at 40° C. and a hydroxyl number of less than 30 mg KOH/g.

In one embodiment, the polyester has a viscosity of at least 200,000 cSt at 40° C. and a hydroxyl number of less than 15 mg KOH/g.

In one embodiment, the polyester has a viscosity of at least 500,000 cSt at 40° C. and a hydroxyl number of less than 15 mg KOH/g.

In one embodiment, the polyester has a viscosity of at least 1,000,000 cSt at 40° C. and a hydroxyl number of less than 15 mg KOH/g.

The invention also relates to a polyester resulting from a reaction between at least one dicarboxylic acid dimer and at least one alcohol carrying at least two hydroxyl functions, the at least one polyester having a viscosity of at least 200,000 cSt at 40° C. and a hydroxyl index of less than 30 mg KOH/g.

In one embodiment, the polyester has a viscosity of at least 500,000 cSt at 40° C. and a hydroxyl number of less than 30 mg KOH/g.

In one embodiment, the polyester has a viscosity of at least 1,000,000 cSt at 40° C. and a hydroxyl number of less than 30 mg KOH/g.

In one embodiment, the polyester has a viscosity of at least 200,000 cSt at 40° C. and a hydroxyl number of less than 15 mg KOH/g.

In one embodiment, the polyester has a viscosity of at least 500,000 cSt at 40° C. and a hydroxyl number of less than 15 mg KOH/g.

In one embodiment, the polyester has a viscosity of at least 1,000,000 cSt at 40° C. and a hydroxyl number of less than 15 mg KOH/g.

In the context of the present invention, the polyester according to the invention is not liquid: it cannot flow under its own weight at 25° C.

The polyester according to the invention has a viscosity of at least 1,000,000 cSt at 40° C. and a hydroxyl number of less than 15 mg KOH/g.

In one embodiment, the polyester according to the invention has a viscosity of at least 1,000,000 cSt at 40° C. and a hydroxyl number of less than 10 mg KOH/g.

In one embodiment, the polyester according to the invention has a viscosity of at least 1,000,000 cSt at 40° C. and a hydroxyl number of less than 5 mg KOH/g.

In one embodiment, the polyester according to the invention has a viscosity greater than or equal to 2,000,000 cSt at 40° C. and a hydroxyl number less than 15 mg KOH/g.

In one embodiment, the polyester according to the invention has a viscosity greater than or equal to 2,000,000 cSt at 40° C. and a hydroxyl number less than 10 mg KOH/g.

In one embodiment, the polyester according to the invention has a viscosity greater than or equal to 2,000,000 cSt at 40° C. and a hydroxyl number less than 5 mg KOH/g.

In one embodiment, the polyester according to the invention has a viscosity greater than or equal to 4,000,000 cSt at 40° C. and a hydroxyl number less than 15 mg KOH/g.

In one embodiment, the polyester according to the invention has a viscosity greater than or equal to 4,000,000 cSt at 40° C. and a hydroxyl number less than 10 mg KOH/g.

In one embodiment, the polyester according to the invention has a viscosity greater than or equal to 4,000,000 cSt at 40° C. and a hydroxyl number less than 5 mg KOH/g.

In one embodiment, the polyester according to the invention has an average molecular weight greater than or equal to 100,000 g/mol.

In one embodiment, the polyester according to the invention has an average molecular weight greater than or equal to 105,000 g/mol.

In one embodiment, the polyester according to the invention has an average molecular weight greater than or equal to 110,000 g/mol.

In one embodiment, the polyester according to the invention has an average molecular weight greater than or equal to 115,000 g/mol.

In one embodiment, the polyester according to the invention has an average molecular weight greater than or equal to 120,000 g/mol.

In one embodiment, the polyester according to the invention has an average molecular weight in the range of 100,000 to 200,000 g/mol.

In one embodiment, the polyester according to the invention has an average molecular weight in the range of 100,000 to 200,000 g/mol.

In one embodiment, the polyester according to the invention has an average molecular weight in the range of 100,000 to 200,000 g/mol.

In one embodiment, the polyester according to the invention has a coloration less than or equal to 200 on the APHA scale.

In one embodiment, the polyester according to the invention has a coloration less than or equal to 175 on the APHA scale.

In one embodiment, the polyester according to the invention has a coloration less than or equal to 150 on the APHA scale.

In one embodiment, the polyester according to the invention has a lower coloration or equal to 125 on the APHA scale.

In one embodiment, the polyester according to the invention has a coloration less than or equal to 100 on the APHA scale.

In one embodiment, the polyester according to the invention has a coloration less than or equal to 75 on the APHA scale.

In a preferred embodiment, the polyester according to the present invention has a lower coloration or equal to 50 on the APHA scale.

The polyester according to the invention is produced from a reaction between at least one dicarboxylic acid dimer and at least one alcohol carrying at least two hydroxyl functions.

In one embodiment, the polyester according to the invention results from a reaction between at least one dicarboxylic acid dimer and at least one alcohol carrying at least two hydroxyl functions, and in the absence of at least one monoalcohol.

In a preferred embodiment, the polyester according to the invention results from a reaction between at least one dicarboxylic acid dimer and at least one alcohol carrying at least two hydroxyl functions, and in the absence of behenyl alcohol and/or phytosterol.

For the purposes of the present invention, the term “reaction” means a condensation reaction.

In one embodiment, the polyester according to the invention is derived from a reaction between at least one dicarboxylic acid dimer of plant origin and at least one alcohol carrying at least two hydroxyl functions of plant origin.

In one embodiment, the polyester according to the invention is derived from a reaction between a dicarboxylic acid dimer of plant origin and an alcohol carrying two hydroxyl functions of plant origin.

In one embodiment, the polyester according to the invention is derived from a reaction between a dicarboxylic acid dimer of plant origin and two alcohols carrying two hydroxyl functions of plant origin.

In one embodiment, the polyester according to the invention results from a reaction between at least one dicarboxylic acid dimer and at least one alcohol carrying two hydroxyl functions.

In one embodiment, the polyester according to the invention results from a reaction between at least one dicarboxylic acid dimer and an alcohol carrying at least two hydroxyl functions.

In one embodiment, the polyester according to the invention results from a reaction between at least one dicarboxylic acid dimer and an alcohol carrying two hydroxyl functions.

In one embodiment, the polyester according to the invention results from a reaction between at least one dicarboxylic acid dimer and at least one alcohol carrying 3 to 8 hydroxyl functions.

In one embodiment, the polyester according to the invention results from a reaction between at least one dicarboxylic acid dimer and an alcohol carrying 3 to 8 hydroxyl functions.

In one embodiment, the polyester according to the invention results from a reaction between at least one dicarboxylic acid dimer and at least two alcohols carrying at least two hydroxyl functions.

In one embodiment, the polyester according to the invention results from a reaction between at least one dicarboxylic acid dimer and at least two alcohols carrying two hydroxyl functions.

In one embodiment, the polyester according to the invention results from a reaction between at least one dicarboxylic acid dimer and two alcohols carrying at least two hydroxyl functions.

In one embodiment, the polyester according to the invention results from a reaction between at least one dicarboxylic acid dimer and two alcohols carrying two hydroxyl functions.

In one embodiment, the polyester according to the invention results from a reaction between a dicarboxylic acid dimer and at least one alcohol carrying two hydroxyl functions.

In one embodiment, the polyester according to the invention results from a reaction between a dicarboxylic acid dimer and an alcohol carrying at least two hydroxyl functions.

In one embodiment, the polyester according to the invention results from a reaction between a dicarboxylic acid dimer and an alcohol carrying two hydroxyl functions.

In one embodiment, the polyester according to the invention results from a reaction between a dicarboxylic acid dimer and at least one alcohol carrying 3 to 8 hydroxyl functions.

In one embodiment, the polyester according to the invention is derived from a reaction between a dicarboxylic acid dimer and an alcohol carrying 3 to 8 hydroxyl functions.

In one embodiment, the polyester according to the invention results from a reaction between a dicarboxylic acid dimer and at least two alcohols carrying at least two hydroxyl functions.

In one embodiment, the polyester according to the invention results from a reaction between a dicarboxylic acid dimer and at least two alcohols carrying two hydroxyl functions.

In one embodiment, the polyester according to the invention results from a reaction between a dicarboxylic acid dimer and two alcohols carrying at least two hydroxyl functions.

In one embodiment, the polyester according to the invention results from a reaction between a dicarboxylic acid dimer and two alcohols carrying two hydroxyl functions.

A dicarboxylic acid dimer is understood to mean a compound obtained by a process comprising a dimerization reaction of a compound bearing a carboxylic acid function.

Thus, the at least one Cₙ dicarboxylic acid dimer is obtained by a process comprising a step of dimerization of a Cₘ compound with n=2×m.

The at least one dicarboxylic acid dimer is a linear or branched, saturated or unsaturated C₈-C₄₄ hydrocarbon compound, which may comprise one or more cycles and carry 2 carboxylic acid functions.

In one embodiment, the dicarboxylic acid dimer is selected from dicarboxylic acid dimers of plant origin.

In one embodiment, the dicarboxylic acid dimer is selected from unsaturated dicarboxylic acid dimers.

In a preferred embodiment, the dicarboxylic acid dimer is chosen from saturated dicarboxylic acid dimers.

In one embodiment, the dicarboxylic acid dimer is a C₂₈-C₄₄ compound.

In one embodiment, the dicarboxylic acid dimer is a C₃₆ compound.

In one embodiment, the dicarboxylic acid dimer is a C₃₆ compound derived from oleic acid, linoleic acid and/or linolenic acid.

In one embodiment, the dicarboxylic acid dimer is a C₃₆ unsaturated compound derived from oleic acid, linoleic acid and/or linolenic acid.

In one embodiment, the dicarboxylic acid dimer is a C₃₆ saturated compound derived from oleic acid, linoleic acid and/or linolenic acid.

In one embodiment, the dicarboxylic acid dimer is a C₃₆ compound selected from the group consisting of products marketed under the trademarks Empol 1008, Pripol 1013, Pripol 1009, Jaric D60, Jaric D70, Jaric D75, Jaric D76, Radiacid 0960, Radiacid 0975, Radiacid 0976, Radiacid 0977 and Radiacid 0978.

In one embodiment, the C₃₆ dicarboxylic acid dimer is the compound of CAS number 68783-41-5.

In one embodiment, the C₃₆ dicarboxylic acid dimer is the product marketed under the brand name Pripol 1009.

It is known that C₃₆ dicarboxylic acid dimer commercial products can also include C₁₈ (“monomer”) and C₃₆ (“trimer”) in small amounts.

In one embodiment, the dicarboxylic acid dimer is a C₄₄ compound.

In one embodiment, the dicarboxylic acid dimer is a C₄₄ unsaturated compound derived from euricic acid.

In one embodiment, the dicarboxylic acid dimer is a C₄₄ saturated compound derived from euricic acid.

In one embodiment, the dicarboxylic acid dimer is a C₄₄ compound selected from the group consisting of products marketed under the trademarks Pripol 1004 and Radiacid 0994.

It is known that C₄₄ dicarboxylic acid dimer products can also include C₂₂ (“monomer”) and C₆₆ (“trimer”) in small amounts.

The at least one alcohol used in the reaction allowing the polyester according to the invention to be obtained is a C₂-C₄₄ hydrocarbon compound, linear or branched, saturated or unsaturated, which may comprise one or more cycles and carry at least 2 hydroxyl functions.

In one embodiment, the at least one alcohol is a saturated linear alcohol.

In one embodiment, the at least one alcohol is a linear unsaturated alcohol.

In one embodiment, the at least one alcohol is a saturated branched alcohol.

In one embodiment, the at least one alcohol is an unsaturated branched alcohol.

In one embodiment, the at least one alcohol is a plant-based alcohol.

In one embodiment, the at least one alcohol is an alcohol bearing two hydroxyl functions.

In one embodiment, the at least one alcohol is an alcohol bearing two hydroxyl functions selected from the group consisting of 1,3-propanediol, 1,2-butanediol, 1,3-butanediol, 1,4-butanediol, 1,5-pentanediol, 1,6-hexanediol and (9Z, 12Z)-18-[(6Z,9Z)-18-hydroxyoctadeca-6,9-dienoxy]octadeca-9,12-dien-1-ol (CAS 147853-32-5), alone or as a mixture.

In one embodiment, the at least one alcohol is an alcohol bearing two hydroxyl functions selected from the group consisting of 1,3-propanediol, 1,4-butanediol, 1,5-pentanediol and (9Z, 12Z)-18-[(6Z, 9Z)-18-hydroxyoctadeca-6,9-dienoxy]octadeca-9,12-dien-1-ol (CAS 147853-32-5).

In one embodiment, the at least one alcohol is 1,3-propanediol.

In one embodiment, the at least one alcohol is 1,4-butanediol.

In one embodiment, the at least one alcohol is 1,5-pentanediol.

In one embodiment, the at least one alcohol is (9Z, 12Z)-18-[(6Z, 9Z)-18-hydroxyoctadeca-6,9-dienoxy]octadeca-9,12-dien-1-ol (CAS 147853-32-5).

In one embodiment, the at least one alcohol carrying two hydroxyl functions is the commercial product of the brand Pripol 2033.

In one embodiment, the at least one alcohol is a mixture of at least two alcohols.

In one embodiment, the at least one alcohol is a mixture of at least two alcohols selected from the group consisting of 1,3-propanediol, 1,2-butanediol, 1,3-butanediol, 1,4-butanediol, 1,5-pentanediol, 1,6-hexanediol and (9Z,12Z)-18-[(6Z,9Z)-18-hydroxyoctadeca-6,9-dienoxy]octadeca-9,12-dien-1-ol (CAS 147853-32-5).

In one embodiment, the at least one alcohol is a mixture of two alcohols.

In one embodiment, the at least one alcohol is a mixture of two linear alcohols.

In one embodiment, the at least one alcohol is a mixture of a linear alcohol and a branched alcohol.

In one embodiment, the at least one alcohol is a mixture of two branched alcohols.

In one embodiment, the at least one alcohol is a mixture of two saturated alcohols.

In one embodiment, the at least one alcohol is a mixture of a saturated alcohol and a saturated alcohol.

In one embodiment, the at least one alcohol is a mixture of two saturated alcohols.

In one embodiment, the at least one alcohol is a mixture of two alcohols selected from the group consisting of 1,3-propanediol, 1,2-butanediol, 1,3-butanediol, 1,4-butanediol, 1,5-pentanediol, 1,6-hexanediol and (9Z,12Z)-18-[(6Z,9Z)-18-hydroxyoctadeca-6,9-dienoxy]octadeca-9,12-dien-1-ol (CAS 147853-32-5).

In one embodiment, the at least one alcohol is a mixture of 1,4-butanediol and (9Z, 12Z)-18-[(6Z, 9Z)-18-hydroxyoctadeca-6,9-dienoxy]octadeca-9,12-dien-1-ol (CAS 147853-32-5).

In one embodiment, the at least one alcohol is selected from the group consisting of alcohols bearing 3 to 8 hydroxyl functions.

In one embodiment, the at least one alcohol is an alcohol bearing 3 to 8 hydroxyl functions selected from the group consisting of glycerol, threitol, erythritol, inositol, arabitol, xylitol and sorbitol.

In one embodiment, the at least one alcohol is a mixture of alcohols carrying 3 to 8 hydroxyl functions.

In one embodiment, the at least one alcohol is a mixture of sorbitol and glycerol.

In one embodiment, the polyester according to the invention results from a reaction between a saturated dicarboxylic acid dimer resulting from oleic acid, linoleic acid and/or linolenic acid and at least one alcohol carrying two hydroxyl functions.

In one embodiment, the polyester according to the invention is derived from a reaction between a saturated dicarboxylic acid dimer derived from oleic acid, linoleic acid and/or linolenic acid and an alcohol carrying at least two hydroxyl functions.

In one embodiment, the polyester according to the invention is derived from a reaction between a saturated dicarboxylic acid dimer derived from oleic acid, linoleic acid and/or linolenic acid and an alcohol carrying two hydroxyl functions.

In one embodiment, the polyester according to the invention is derived from a reaction between a saturated dicarboxylic acid dimer derived from oleic acid, linoleic acid and/or linolenic acid and (9Z, 12Z)-18-[(6Z, 9Z)-18-hydroxyoctadeca-6,9-dienoxy]octadeca-9,12-dien-1-ol (CAS 147853-32-5).

In one embodiment, the polyester according to the invention is derived from a reaction between a saturated dicarboxylic acid dimer derived from oleic acid, linoleic acid and/or linolenic acid and 1,3-propanediol.

In one embodiment, the polyester according to the invention is derived from a reaction between a saturated dicarboxylic acid dimer derived from oleic acid, linoleic acid and/or linolenic acid and 1,4-propanediol.

In one embodiment, the polyester according to the invention is derived from a reaction between a saturated dicarboxylic acid dimer derived from oleic acid, linoleic acid and/or linolenic acid and 1,5-pentanediol.

In one embodiment, the polyester according to the invention is derived from a reaction between a saturated dicarboxylic acid dimer derived from oleic acid, linoleic acid and/or linolenic acid and at least one alcohol carrying 3 to 8 hydroxyl functions.

In one embodiment, the polyester according to the invention is derived from a reaction between a saturated dicarboxylic acid dimer derived from oleic acid, linoleic acid and/or linolenic acid and an alcohol carrying 3 to 8 hydroxyl functions.

In one embodiment, the polyester according to the invention results from a reaction between a saturated dicarboxylic acid dimer resulting from oleic acid, linoleic acid and/or linolenic acid and at least two alcohols carrying at least two hydroxyl functions.

In one embodiment, the polyester according to the invention is derived from a reaction between a saturated dicarboxylic acid dimer derived from oleic acid, linoleic acid and/or linolenic acid and at least two alcohols carrying two hydroxyl functions.

In one embodiment, the polyester according to the invention results from a reaction between a saturated dicarboxylic acid dimer resulting from oleic acid, linoleic acid and/or linolenic acid and two alcohols carrying at least two hydroxyl functions.

In one embodiment, the polyester according to the invention is derived from a reaction between a saturated dicarboxylic acid dimer derived from oleic acid, linoleic acid and/or linolenic acid and two alcohols carrying two hydroxyl functions.

In one embodiment, the polyester according to the invention is derived from a reaction between a saturated dicarboxylic acid dimer derived from oleic acid, linoleic acid and/or linolenic acid and 1,4-butanediol and (9Z, 12Z)-18-[(6Z, 9Z)-18-hydroxyoctadeca-6,9-dienoxy]octadeca-9,12-dien-1-ol (CAS 147853-32-5).

The term “biosourced hydrocarbon compound” means a compound formed essentially, or even consisting of, carbon and hydrogen atoms, and possibly oxygen and nitrogen atoms, characterized in that the organic carbon present in the compound is 100% of plant origin (¹⁴C) by radiocarbon analysis according to one of the following standards: ASTM D6866, EN 16640 or EN 16785-1.

In a preferred embodiment, the at least one biosourced hydrocarbon compound present in the mixture according to the invention consists only of carbon and hydrogen atoms.

The at least one biosourced hydrocarbon compound present in the mixture according to the invention comprises at least 12 carbon atoms.

In one embodiment, the at least one biosourced hydrocarbon compound present in the mixture according to the invention comprises at least 16 carbon atoms.

In one embodiment, the at least one biosourced hydrocarbon compound present in the mixture according to the invention comprises at least 22 carbon atoms.

In one embodiment, the at least one biosourced hydrocarbon compound present in the mixture according to the invention comprises at least 30 carbon atoms.

In one embodiment, the mixture according to the invention comprises a biosourced hydrocarbon compound.

In one embodiment, the mixture according to the invention comprises two biosourced hydrocarbon compounds.

In one embodiment, the at least one biosourced hydrocarbon compound present in the mixture according to the invention is chosen from the group consisting of linear or branched, saturated or unsaturated alkanes.

In one embodiment, the at least one biosourced hydrocarbon compound present in the mixture according to the invention is chosen from the group consisting of saturated linear alkanes.

In one embodiment, the at least one biosourced hydrocarbon compound present in the mixture according to the invention is chosen from the group consisting of saturated branched alkanes.

In one embodiment, the at least one biosourced hydrocarbon compound present in the mixture according to the invention is chosen from the group consisting of unsaturated linear alkanes.

In one embodiment, the at least one biosourced hydrocarbon compound present in the mixture according to the invention is chosen from the group consisting of unsaturated branched alkanes.

In one embodiment, the at least one biosourced hydrocarbon compound present in the mixture according to the invention is a non-volatile compound.

In one embodiment, the at least one biosourced hydrocarbon compound present in the mixture according to the invention has a flash point greater than 110° C. measured according to the ATSM D93 standard.

In one embodiment, the at least one biosourced hydrocarbon compound present in the mixture according to the invention is chosen from the group consisting of squalane and squalene.

In one embodiment, the at least one biosourced hydrocarbon compound present in the mixture according to the invention is squalane.

In one embodiment, the at least one biosourced hydrocarbon compound present in the mixture according to the invention is squalene.

In one embodiment, the at least one biosourced hydrocarbon compound present in the mixture according to the invention is a mixture of alkanes.

In one embodiment, the at least one biosourced hydrocarbon compound present in the mixture according to the invention is a mixture of C₁₆-C₁₈ alkanes.

Advantageously, the at least one biosourced hydrocarbon compound present in the mixture according to the invention has a biodegradability of at least 60% according to the OECD 301F method.

In one embodiment, the mixture according to the invention is characterized in that the mass percentage of the at least one polyester is at least 50% relative to the total mass of the mixture.

In one embodiment, the mixture according to the invention is characterized in that the mass percentage of the at least one polyester is between 50 and 95% relative to the total mass of the mixture.

In one embodiment, the mixture according to the invention is characterized in that the mass percentage of the at least one polyester is between 50 and 70% relative to the total mass of the mixture.

In one embodiment, the mixture according to the invention is characterized in that the mass percentage of the at least one polyester is between 60 and 80% relative to the total mass of the mixture.

In one embodiment, the mixture according to the invention is characterized in that the mass percentage of the at least one polyester is between 70 and 90% relative to the total mass of the mixture.

In one embodiment, the mixture according to the invention is characterized in that the mass percentage of the at least one polyester is between 75 and 95% relative to the total mass of the mixture.

In one embodiment, the mixture according to the invention is characterized in that the mass percentage of the at least one biosourced hydrocarbon compound is at least 5% relative to the total mass of the mixture.

In one embodiment, the mixture according to the invention is characterized in that the mass percentage of the at least one biosourced hydrocarbon compound is between 5 and 50% relative to the total mass of the mixture.

In one embodiment, the mixture according to the invention is characterized in that the mass percentage of the at least one biosourced hydrocarbon compound is between 5 and 15% relative to the total mass of the mixture.

In one embodiment, the mixture according to the invention is characterized in that the mass percentage of the at least one biosourced hydrocarbon compound is between 15 and 25% relative to the total mass of the mixture.

In one embodiment, the mixture according to the invention is characterized in that the mass percentage of the at least one biosourced hydrocarbon compound is between 25 and 35% relative to the total mass of the mixture.

In one embodiment, the mixture according to the invention is characterized in that the mass percentage of the at least one biosourced hydrocarbon compound is between 35 and 50% relative to the total mass of the mixture.

In one embodiment, the mixture according to the invention is characterized in that it comprises at least 50% by mass of the at least one polyester and at least 10% by mass of the at least one biosourced hydrocarbon compound.

In one embodiment, the mixture according to the invention is characterized in that it comprises at least 50% by mass of the at least one polyester and at least 20% by mass of the at least one biosourced hydrocarbon compound.

In one embodiment, the mixture according to the invention is characterized in that it comprises at least 50% by mass of the at least one polyester and at least 30% by mass of the at least one biosourced hydrocarbon compound.

In one embodiment, the mixture according to the invention is characterized in that it comprises at least 60% by mass of the at least one polyester and at least 10% by mass of the at least one biosourced hydrocarbon compound.

In one embodiment, the mixture according to the invention is characterized in that it comprises at least 70% by mass of the at least one polyester and at least 10% by mass of the at least one biosourced hydrocarbon compound.

In one embodiment, the mixture according to the invention is characterized in that it contains 50% by mass of at least one polyester and 50% by mass of at least one biosourced hydrocarbon compound.

In one embodiment, the mixture according to the invention is characterized in that it contains 60% by mass of at least one polyester and 40% by mass of at least one biosourced hydrocarbon compound.

In one embodiment, the mixture according to the invention is characterized in that it contains 70% by mass of at least one polyester and 30% by mass of at least one biosourced hydrocarbon compound.

In one embodiment, the mixture according to the invention is characterized in that it contains 80% by mass of at least one polyester and 20% by mass of at least one biosourced hydrocarbon compound.

The invention also relates to a process for obtaining the mixture according to the invention, characterized in that it comprises the following steps:
a) at least one carboxylic acid dimer is available,
b) at least one alcohol carrying at least two hydroxyl functions is available,
c) at least one biosourced hydrocarbon compound is available,
d) the compounds from steps a), b) and c) are mixed,
e) the pressure of the mixture is lowered to a pressure between 900 and 10 mbar,
f) the mixture is gradually heated to a target temperature between 150° C. and 250° C.,
g) the water formed during the condensation reaction is continuously removed,
h) a mixture is obtained comprising a polyester and the biosourced hydrocarbon compound,
   characterized in that the compounds of steps a), b) and c) can be mixed in any order during step d).

The invention also relates to a process for obtaining the polyester according to the invention, characterized in that it comprises the following steps:
a) at least one carboxylic acid dimer is available,
b) at least one alcohol carrying at least two hydroxyl functions is available,
c) the compounds from steps a), b) and c) are mixed,
d) the pressure of the mixture is lowered to a pressure between 900 and 10 mbar,
e) the mixture is gradually heated to a target temperature between 150° C. and 250° C.,
f) the water formed during the condensation reaction is continuously removed,
g) a polyester is obtained,
   characterized in that the compounds of steps a) and b) can be mixed in any order during step d).

In one embodiment, the method for obtaining the mixture according to the invention is characterized in that it does not use monoalcohol.

In a preferred embodiment, the process for obtaining the mixture according to the invention is characterized in that it does not use behenyl alcohol and/or phytosterol.

In one embodiment, at least one catalyst is added during step d) of the process for obtaining the mixture according to the invention.

In one embodiment, the at least one catalyst added during step d) is selected from the group consisting of base type catalysts.

In one embodiment, the at least one catalyst added during step d) is a base-type catalyst selected from the group consisting of hydroxide or carbonate salts of alkali metals such as sodium hydroxide, potassium hydroxide and potassium carbonate.

In one embodiment, the at least one catalyst added during step d) is selected from the group consisting of acid-type catalysts.

In one embodiment, the at least one catalyst added during step d) is an acid-type catalyst selected from the group consisting of sulfonic acids such as paratoluenesulfonic acid, methanesulfonic acid and phosphoric or oxy-phosphoric acids such as phosphoric acid and phosphorous acid.

In a preferred embodiment, the at least one catalyst added during step d) is methanesulfonic acid.

In one embodiment, the at least one catalyst added during step d) is selected from the group consisting of tin oxalate and titanium tetrabutanolate.

In a preferred embodiment, the at least one catalyst added during step d) is tin oxalate.

In one embodiment, the mass of the at least one catalyst added during step d) is between 0.01 and 1% relative to the sum of the masses of the at least one dicarboxylic acid dimer and the at least one alcohol.

In one embodiment, the mass of the at least one catalyst added during step d) is between 0.01 and 0.5% relative to the sum of the masses of the at least one dicarboxylic acid dimer and the at least one alcohol.

In one embodiment, the mass of the at least one catalyst added during step d) is between 0.01 and 0.2% relative to the sum of the masses of the at least one dicarboxylic acid dimer and the at least one alcohol.

In one embodiment, the mass of the at least one catalyst added during step d) is between 0.01 and 0.1% relative to the sum of the masses of the at least one dicarboxylic acid dimer and the at least one alcohol.

In one embodiment, the pressure is lowered to a pressure between 10 mbar and 900 mbar during step e) of the process for obtaining the mixture according to the invention.

In one embodiment, the pressure is lowered to a pressure between 10 mbar and 600 mbar during step e) of the process for obtaining the mixture according to the invention.

In one embodiment, the pressure is lowered to a pressure between 10 mbar and 300 mbar during step e) of the process for obtaining the mixture according to the invention.

In one embodiment, the pressure is lowered to a pressure of 10 mbar during step e) of the process for obtaining the mixture according to the invention.

In one embodiment, the pressure is lowered to a pressure of 200 mbar during step e) of the process for obtaining the mixture according to the invention.

In one embodiment, the mixture is gradually heated to a target temperature during step f) of the method for obtaining the mixture according to the invention by following a temperature ramp.

In one embodiment, the mixture is gradually heated to a target temperature during step f) of the process for obtaining the mixture according to the invention by carrying out temperature steps.

In one embodiment, the mixture is gradually heated to a temperature between 150 and 170° C. during step f) of the process for obtaining the mixture.

In one embodiment, the mixture is gradually heated to a temperature between 170 and 190° C. during step f) of the process for obtaining the mixture according to the invention.

In one embodiment, the mixture is gradually heated to a temperature between 190 and 210° C. during step f) of the process for obtaining the mixture according to the invention.

In one embodiment, the mixture is gradually heated to a temperature between 210 and 230° C. during step f) of the process for obtaining the mixture according to the invention.

In one embodiment, the mixture is gradually heated to a temperature between 230 and 250° C. during step f) of the process for obtaining the mixture according to the invention.

In one embodiment, the mixture is gradually heated to a temperature of 160° C. during step f) of the process for obtaining the mixture according to the invention.

In one embodiment, the mixture is gradually heated to a temperature of 170° C. during step f) of the process for obtaining the mixture according to the invention.

In one embodiment, the mixture is gradually heated to a temperature of 180° C. during step f) of the process for obtaining the mixture according to the invention.

In one embodiment, the mixture is gradually heated to a temperature of 190° C. during step f) of the process for obtaining the mixture according to the invention.

In one embodiment, the mixture is gradually heated to a temperature of 200° C. during step f) of the process for obtaining the mixture according to the invention.

In one embodiment, the mixture is gradually heated to a temperature of 210° C. during step f) of the process for obtaining the mixture according to the invention.

In one embodiment, the mixture is gradually heated to a temperature of 220° C. during step f) of the process for obtaining the mixture according to the invention.

In one embodiment, the mixture is gradually heated to a temperature of 230° C. during step f) of the process for obtaining the mixture according to the invention.

In one embodiment, the excess of the at least one alcohol carrying at least two hydroxyl functions is also eliminated during step g) of the process for obtaining the mixture according to the invention.

In one embodiment, the process for obtaining the mixture according to the invention is characterized in that the alcohol carrying at least two hydroxyl functions is a diol.

In one embodiment, the method for obtaining the mixture according to the invention is characterized in that it comprises the following steps:
a) a dicarboxylic acid dimer is available,
b) a diol is available,
c) a biosourced hydrocarbon compound is available,
d) mixing the compounds from steps a), b) and c),
e) the pressure of the mixture is reduced to a pressure between 900 and 10 mbar
f) the mixture is gradually heated to a target temperature between 150 and 250° C.,
g) the water formed during the condensation reaction is continuously removed,
h) a mixture is obtained comprising a polyester and the biosourced hydrocarbon compound,
   characterized in that the compounds of steps a), b) and c) can be mixed in any order during step d).

In one embodiment, the method for obtaining the mixture according to the invention is characterized in that it comprises the following steps:
a) a dicarboxylic acid dimer is available,
b) a diol is available,
c) a biosourced hydrocarbon compound is available,
d) mixing the compounds from steps a), b) and c),
e) the pressure of the mixture is reduced to a pressure between 900 and 10 mbar,
f) the mixture is gradually heated to a target temperature between 150 and 250° C.,
g) the water formed during the condensation reaction is continuously removed,
h) a mixture is obtained comprising a polyester and the biosourced hydrocarbon compound,
   characterized in that a catalyst is added during step d).

In one embodiment, the method for obtaining the mixture according to the invention is characterized in that it comprises the following steps:
a) a saturated dicarboxylic acid dimer is available from oleic acid, linoleic acid and/or linolenic acid,
b) (9Z,12Z)-18-[(6Z,9Z)-18-hydroxyoctadeca-6,9-dienoxy]octadeca-9,12-dien-1-ol (CAS 147853-32-5) is available,
c) squalane is available,
d) mixing the compounds from steps a), b) and c),
e) the pressure of the mixture is reduced to a pressure of 10 mbar,
f) the mixture is gradually heated to a temperature of 220° C.,
g) the water formed during the condensation reaction is continuously removed,
h) a mixture is obtained comprising a polyester and squalane.

In one embodiment, the method for obtaining the mixture according to the invention is characterized in that it comprises the following steps:
a) a saturated dicarboxylic acid dimer is available from oleic acid, linoleic acid and/or linolenic acid
b) (9Z,12Z)-18-[(6Z,9Z)-18-hydroxyoctadeca-6,9-dienoxy]octadeca-9,12-dien-1-ol (CAS 147853-32-5) is available,
c) squalene is available,
d) mixing the compounds from steps a), b) and c),
e) the pressure of the mixture is reduced to a pressure of 10 mbar,
f) the mixture is gradually heated to a temperature of 160° C.,
g) the water formed during the condensation reaction is continuously removed,
methanesulfonic acid being added in step d) and
h) a mixture is obtained comprising a polyester and squalene.

In one embodiment, the method for obtaining the mixture according to the invention is characterized in that it comprises the following steps:
a) a saturated dicarboxylic acid dimer from oleic acid, linoleic acid and/or linolenic acid is available,
b) (9Z,12Z)-18-[(6Z,9Z)-18-hydroxyoctadeca-6,9-dienoxy]octadeca-9,12-dien-1-ol (CAS 147853-32-5) is available,
c) a mixture of C₁₆-C₁₈ alkanes is available,
d) mixing the compounds from steps a), b) and c),
e) the pressure of the mixture is reduced to a pressure of 200 mbar,
f) the mixture is gradually heated to a temperature of 170° C.,
g) the water formed during the condensation reaction is continuously removed,
oxalate being added in step d) and
h) a mixture is obtained comprising a polyester and the C₁₆-C₁₈ alkanes.

The invention also relates to a cosmetic composition comprising a mixture according to the invention and at least one compound chosen from non-volatile oils, volatile oils, glossy oils, pasty fatty substances, gelling agents, film-forming polymers, waxes, antioxidants, pigments, dyes, surfactants, an aqueous phase, and mixtures thereof.

In one embodiment, the cosmetic composition according to the invention is characterized in that it comprises a mixture according to the invention and at least one non-volatile oil.

For the purposes of the present invention, the term “non-volatile oil” means an oil having a flash point greater than 110° C. measured according to the ATSM D93 standard.

These oils can be of vegetable, mineral or synthetic origin.

These non-volatile oils can be hydrocarbon, silicone, fluorinated, or fluorosilicone oils.

For the purposes of the present invention, the term “hydrocarbon oil” means an oil essentially consisting of carbon atoms, hydrogen atoms and optionally oxygen and/or nitrogen atoms.

Among these hydrocarbon oils, mention may be made of linear or branched alkanes of mineral or synthetic origin such as linear or branched alkanes of 12 to 40 carbon atoms, (poly)esters and (poly)ethers, and in particular (poly)esters of C₂-C₂₄ acids (preferably C₆-C₂₀), triglycerides of C₆-C₂₀ fatty acids, vegetable oils, dialkyl carbonates, branched and/or unsaturated fatty acids, branched and/or unsaturated fatty alcohols.

Concerning non-volatile silicone oils, they are notably chosen from the group comprising phenylated silicone oils, non-volatile polydimethylsiloxanes, or derivatives of non-volatile polydimethylsiloxanes and their mixtures.

In a particularly preferred embodiment, the cosmetic composition according to the invention is characterized in that the non-volatile oil is chosen from the group consisting of non-volatile linear or branched alkanes.

Preferably, the non-volatile oil is chosen from the group consisting of linear or branched alkanes of 12 to 40 carbon atoms.

Preferably, non-volatile oils are of plant origin.

In a preferred embodiment, the cosmetic composition according to the invention is characterized in that the non-volatile oil is a vegetable hydrocarbon oil.

The vegetable hydrocarbon oils are notably chosen from the group comprising wheat germ, sunflower, grape seed, sesame, coconut, apricot, castor, corn, avocado, soybean, shea, olive, sweet almond oil, cotton, palm, macadamia, rapeseed, jojoba, hazelnut, poppy, alfalfa, pumpkin, blackcurrant, squash, evening primrose, barley oils, and mixtures thereof.

In one embodiment, the cosmetic composition according to the invention is characterized in that the mass percentage of the non-volatile oil is at least 5% relative to the total mass of the cosmetic composition.

In one embodiment, the cosmetic composition according to the invention is characterized in that the mass percentage of the non-volatile oil is at least 10% relative to the total mass of the cosmetic composition.

In one embodiment, the cosmetic composition according to the invention is characterized in that the mass percentage of the non-volatile oil is between 5% and 25% relative to the total mass of the cosmetic composition.

In one embodiment, the cosmetic composition according to the invention is characterized in that the mass percentage of the non-volatile oil is between 5% and 15% relative to the total mass of the cosmetic composition.

In one embodiment, the cosmetic composition according to the invention is characterized in that the mass percentage of the non-volatile oil is between 10% and 25% relative to the total mass of the cosmetic composition.

In one embodiment, the cosmetic composition according to the invention is characterized in that it comprises a mixture according to the invention and at least one volatile oil.

For the purposes of the present invention, the term “volatile oil” means an oil having a flash point below 110° C. measured according to the ATSM D93 standard.

These oils can be of vegetable, mineral or synthetic origin.

In a preferred embodiment, the cosmetic composition according to the invention is characterized in that it comprises a mixture according to the invention and at least one volatile oil of plant origin.

In one embodiment, the at least one volatile oil has a flash point below 95° C. measured according to ATSM D93.

In one embodiment, the at least one volatile oil has a flash point below 55° C. measured according to ATSM D93.

In one embodiment, the at least one volatile oil has a flash point between 40 and 55° C. measured according to the ATSM D93 standard.

In one embodiment, the at least one volatile oil has a flash point between 30 and 40° C. measured according to the ATSM D93 standard.

These volatile oils can be hydrocarbon, silicone, fluorinated, or fluorosilicone oils fluorosilicone oils.

Among these hydrocarbon oils, we can cite linear or branched C₈-C₁₆ alkanes, in particular C₈-C₁₆ isoalkanes such as isodecane, isododecane and isohexadecane.

In one embodiment, the cosmetic composition according to the invention is characterized in that it comprises a mixture according to the invention and at least one volatile oil chosen from the group consisting of branched C₈-C₁₂ alkanes.

In one embodiment, the cosmetic composition according to the invention is characterized in that it comprises a mixture according to the invention and at least one volatile oil chosen from the group consisting of branched C₁₀ alkanes.

In one embodiment, the cosmetic composition according to the invention is characterized in that it comprises a mixture according to the invention and at least one volatile oil chosen from the group consisting of trimethylheptanes.

For the purpose of the present invention “trimethylheptanes” means all C₇ alkanes branched by three methyl groups.

In one embodiment, the cosmetic composition according to the invention is characterized in that it comprises a mixture according to the invention and at least one volatile oil chosen from the group consisting of branched C₁₀ alkanes and linear C>10 alkanes.

In one embodiment, the cosmetic composition according to the invention is characterized in that it comprises a mixture according to the invention and at least one volatile oil chosen from the group consisting of trimethylheptanes and linear C>10 alkanes.

In one embodiment, the cosmetic composition according to the invention is characterized in that it comprises a mixture according to the invention and at least one volatile oil consisting of a mixture of at least one trimethylheptane and at least one linear C>10 alkane.

Among these hydrocarbon oils, we can also cite branched C₈-C₁₆ esters such as isohexyl neopentanoate.

Among these silicone oils, we can cite linear or cyclic silicone oils such as linear or cyclic polydimethylsiloxanes having 3 to 7 silicon atoms.

Among these fluorinated oils, we can cite fluorinated oils such as perfluoropolyethers, perfluoroalkanes such as perfluorodecalin, perfluorodamantanes and fluorinated ester oils.

In one embodiment, the cosmetic composition according to the invention is characterized in that the mass percentage of the volatile oil is at least 5% relative to the total mass of the cosmetic composition.

In one embodiment, the cosmetic composition according to the invention is characterized in that the mass percentage of the volatile oil is at least 10% relative to the total mass of the cosmetic composition.

In one embodiment, the cosmetic composition according to the invention is characterized in that the mass percentage of the volatile oil is at least 15% relative to the total mass of the cosmetic composition.

In one embodiment, the cosmetic composition according to the invention is characterized in that the mass percentage of the volatile oil is at least 20% relative to the total mass of the cosmetic composition.

In one embodiment, the cosmetic composition according to the invention is characterized in that the mass percentage of the volatile oil is at least 25% relative to the total mass of the cosmetic composition.

In one embodiment, the cosmetic composition according to the invention is characterized in that the mass percentage of the volatile oil is at least 50% relative to the total mass of the cosmetic composition.

In one embodiment, the cosmetic composition according to the invention is characterized in that the mass percentage of the volatile oil is between 25% and 80% relative to the total mass of the cosmetic composition.

In one embodiment, the cosmetic composition according to the invention is characterized in that the mass percentage of the volatile oil is between 40% and 80% relative to the total mass of the cosmetic composition.

In one embodiment, the cosmetic composition according to the invention is characterized in that the mass percentage of the volatile oil is between 25% and 60% relative to the total mass of the cosmetic composition.

In one embodiment, the cosmetic composition according to the invention is characterized in that the mass percentage of the volatile oil is between 30% and 50% relative to the total mass of the cosmetic composition.

In one embodiment, the cosmetic composition according to the invention is characterized in that the mass percentage of the volatile oil is between 40% and 60% relative to the total mass of the cosmetic composition.

In one embodiment, the cosmetic composition according to the invention is characterized in that the mass percentage of the volatile oil is between 60% and 80% relative to the total mass of the cosmetic composition.

In one embodiment, the cosmetic composition according to the invention is characterized in that it comprises a mixture according to the invention and at least one shiny oil.

For the purposes of the present invention, the term “shiny oil” means an oil having intrinsic shine properties. “Shiny oils” are frequently used in cosmetics to ensure a “shiny effect” when applying cosmetic compositions to keratin materials, for example.

In one embodiment, the cosmetic composition according to the invention is characterized: the shiny oil is a non-volatile oil.

In one embodiment, the cosmetic composition according to the invention is characterized in that the shiny oil is chosen from the group comprising:
polymers such as: polybutylenes, in particular POLYBUTENE® marketed or manufactured by the company ATAMAN, hydrogenated polyisobutylenes, polydecenes and hydrogenated polydecenes, polyfarnesenes, vinylpyrrolidone copolymers, in particular the vinylpyrrolidone/eicosene copolymer, ANTARON V 220® marketed or manufactured by the company ISP.
esters such as esters of linear fatty acids having a total number of carbons greater than 30, aromatic esters, esters of fatty alcohols or branched fatty acids having from 20 to 30 carbon atoms.
ester copolymers such as: dilinoleyl dimers/dilinoleate dimers (LUSPLAN DD-DA5® and LUSPLAN DD-DA7®), dilinoleic acid/butanediol copolymer (VISCOPLAST 14436H®), dilinoleic acid/propanediol copolymer and their mixtures.

Preferably, the cosmetic composition according to the invention is characterized in that the shiny oil is of plant origin.

In one embodiment, the cosmetic composition according to the invention is characterized in that the mass percentage of the shiny oil is at least 2% relative to the total mass of the cosmetic composition.

In one embodiment, the cosmetic composition according to the invention is characterized in that the mass percentage of the shiny oil is at least 5% relative to the total mass of the cosmetic composition.

In one embodiment, the cosmetic composition according to the invention is characterized in that the mass percentage of the shiny oil is between 2% and 20% relative to the total mass of the cosmetic composition.

In one embodiment, the cosmetic composition according to the invention is characterized in that the mass percentage of the shiny oil is between 5% and 15% relative to the total mass of the cosmetic composition.

In one embodiment, the cosmetic composition according to the invention is characterized in that the mass percentage of the shiny oil is between 10% and 20% relative to the total mass of the cosmetic composition.

In one embodiment, the cosmetic composition according to the invention is characterized in that the mass percentage of the shiny oil is between 2% and 10% relative to the total mass of the cosmetic composition.

In one embodiment, the cosmetic composition according to the invention is characterized in that the mass percentage of the shiny oil is between 5% and 10% relative to the total mass of the cosmetic composition.

In one embodiment, the cosmetic composition according to the invention is characterized in that it comprises a mixture according to the invention and at least one pasty fatty substance.

For the purposes of the present invention, the term “pasty fatty substance” means a lipophilic fatty compound with a reversible solid/liquid state change, having at room temperature (25° C.) a liquid fraction and a solid fraction.

In other words, the melting temperature of the pasty fat is less than or equal to 25° C.

In one embodiment, the cosmetic composition according to the invention is characterized in that the pasty fatty substance is chosen from the group comprising synthetic pasty fatty substances, animal pasty fatty substances, vegetable pasty fatty substances and their mixtures.

In one embodiment, the cosmetic composition according to the invention is characterized in that the pasty fatty substance is a synthetic pasty fatty substance chosen from the group comprising: polyol ethers, petroleum jelly, vinyl polymers, pentaerythritol esters, polyesters and their mixtures.

In one embodiment, the cosmetic composition according to the invention is characterized in that the animal pasty fatty substance is lanolin.

Preferably, the cosmetic composition according to the invention is characterized in that the pasty fatty substance is a vegetable pasty fatty substance.

In one embodiment, the cosmetic composition according to the invention is characterized in that the vegetable pasty fatty body is chosen from the group comprising: vegetable butters (avocado butter, shea butter), fatty acid triglycerides and their derivatives, and their mixtures.

In one embodiment, the cosmetic composition according to the invention is characterized in that the mass percentage of the pasty fatty substance is between 0% and 20% relative to the total mass of the cosmetic composition.

In one embodiment, the cosmetic composition according to the invention is characterized in that the mass percentage of the pasty fatty substance is between 0% and 15% relative to the total mass of the cosmetic composition.

In one embodiment, the cosmetic composition according to the invention is characterized in that the mass percentage of the pasty fatty substance is between 0% and 10% relative to the total mass of the cosmetic composition.

In one embodiment, the cosmetic composition according to the invention is characterized in that the mass percentage of the pasty fatty substance is between 0% and 5% relative to the total mass of the cosmetic composition.

In one embodiment, the cosmetic composition according to the invention is characterized in that it comprises a mixture according to the invention and at least one gelling agent.

For the purposes of the present invention, the term gelling agent means compounds which constitute excellent texturizing agents for balancing formulations in terms of smoothness and stickiness.

Furthermore, certain glossy oils such as VISCOPLAST 14436 HR, and compounds marketed under the names LUSPLAN DD-DA5® and LUSPLAN DD-DA7® can also be used as gelling agents.

In one embodiment, the cosmetic composition according to the invention is characterized in that the gelling agent is chosen from the group comprising: organic gelling agents, mineral gelling agents, polymeric gelling agents, and mixtures thereof.

Preferably, the cosmetic composition according to the invention is characterized in that the gelling agent is of plant origin.

In one embodiment, the cosmetic composition according to the invention is characterized in that the gelling agent is an organic gelling agent chosen from the group comprising: ethylcellulose and its derivatives, resins derived from rosin, dextrin esters, fatty acid esters, and mixtures thereof.

In one embodiment, the cosmetic composition according to the invention is characterized in that the gelling agent is a mineral gelling agent chosen from the group comprising: hectorite and its derivatives, pyrogenic silica and its derivatives, and their mixtures.

In one embodiment, the cosmetic composition according to the invention is characterized in that the gelling agent is a polymeric gelling agent chosen from the group comprising: organopolysiloxanes and their derivatives, dimethicone copolymers and their derivatives, polystyrene/polyisoprene copolymers, polystyrene/polybutadiene copolymers, polystyrene/copoly(ethylene-propylene) copolymers, polystyrene/copoly(ethylene-butylene) copolymers, mixtures of copolymers such in isododecane as ethylene/propylene copolymer or butylene/ethylene/styrene copolymer, polyesters and their derivatives, polyamide resins, and mixtures thereof.

In one embodiment, the cosmetic composition according to the invention is characterized in that the gelling agent is dextrin palmitate or one of its derivatives.

In one embodiment, the cosmetic composition according to the invention is characterized in that the gelling agent is a modified hectorite of the BENTONE GEL ISD V® type or one of its equivalents.

In one embodiment, the cosmetic composition according to the invention is characterized in that the gelling agent is a modified hectorite of the VEGELIGHT BENTONE SILK SB® type or one of its equivalents.

In one embodiment, the cosmetic composition according to the invention is characterized in that the gelling agent is a modified hectorite of the disteardimonium hectorite type associated with triethyl citrate.

In one embodiment, the cosmetic composition according to the invention is characterized in that the gelling agent is a mixture of copolymer in isododecane such as ethylene/propylene copolymer.

In one embodiment, the cosmetic composition according to the invention is characterized in that the gelling agent is the combination of a dimethicone polymer and a mixture of alkanes marketed under the name VEGELIGHT SILK SI 118® by BIOSYNTHIS, or one of its equivalents.

In one embodiment, the cosmetic composition according to the invention is characterized in that the mass percentage of the gelling agent is at least 5% relative to the total mass of the cosmetic composition.

In one embodiment, the cosmetic composition according to the invention is characterized in that the mass percentage of the gelling agent is at least 15% relative to the total mass of the cosmetic composition.

In one embodiment, the cosmetic composition according to the invention is characterized in that the mass percentage of the gelling agent is between 5% and 30% relative to the total mass of the cosmetic composition.

In one embodiment, the cosmetic composition according to the invention is characterized in that the mass percentage of the gelling agent is between 5% and 20% relative to the total mass of the cosmetic composition.

In one embodiment, the cosmetic composition according to the invention is characterized in that the mass percentage of the gelling agent is between 15% and 30% relative to the total mass of the cosmetic composition.

In one embodiment, the cosmetic composition according to the invention is characterized in that the mass percentage of the gelling agent is between 5% and 15% relative to the total mass of the cosmetic composition.

In one embodiment, the cosmetic composition according to the invention is characterized in that the mass percentage of the gelling agent is between 15% and 25% relative to the total mass of the cosmetic composition.

In one embodiment, the cosmetic composition according to the invention is characterized in that the mass percentage of the gelling agent is between 20% and 30% relative to the total mass of the cosmetic composition.

In one embodiment, the cosmetic composition according to the invention is characterized in that it comprises a mixture according to the invention and at least one film-forming polymer.

For the purposes of the present invention, the term film-forming polymer means a polymer capable of forming a macroscopically continuous film, in particular on the lips within the framework of our invention.

In one embodiment, the cosmetic composition according to the invention is characterized in that the film-forming polymer is chosen from the group comprising: acrylic polymers, polyurethanes, polyesters, polyamides, silicone polymers.

In one embodiment, the cosmetic composition according to the invention is characterized in that the film-forming polymer is trimethylsilloxysilicate marketed under the name TMS 803.

In one embodiment, the cosmetic composition according to the invention is characterized in that the mass percentage of the film-forming polymer is at least 1% relative to the total mass of the cosmetic composition.

In one embodiment, the cosmetic composition according to the invention is characterized in that the mass percentage of the film-forming polymer is at least 10% relative to the total mass of the cosmetic composition.

In one embodiment, the cosmetic composition according to the invention is characterized in that the mass percentage of the film-forming polymer is between 1% and 25% relative to the total mass of the cosmetic composition.

In one embodiment, the cosmetic composition according to the invention is characterized in that the mass percentage of the film-forming polymer is between 10% and 25% relative to the total mass of the cosmetic composition.

In one embodiment, the cosmetic composition according to the invention is characterized in that the mass percentage of the film-forming polymer is between 1% and 10% relative to the total mass of the cosmetic composition.

In one embodiment, the cosmetic composition according to the invention is characterized in that the mass percentage of the film-forming polymer is between 10% and 20% relative to the total mass of the cosmetic composition.

In one embodiment, the cosmetic composition according to the invention is characterized in that it comprises a mixture according to the invention and at least one wax.

Waxes play an important role in the design of cosmetic products, particularly lipsticks, because they influence certain essential criteria such as the texture, appearance and/or solidity of the product.

In the context of the present invention, the term “wax” means a lipophilic compound having a reversible solid/liquid state change. In other words, the compound is solid at room temperature (at 25° C.) and has a melting point greater than or equal to 30° C.

Preferably, for the purposes of the present invention, the waxes used have a melting point greater than or equal to 50° C.

In one embodiment, the cosmetic composition according to the invention is characterized in that the at least one wax is chosen from the group consisting of synthetic waxes, mineral waxes, vegetable waxes and animal waxes.

In one embodiment, the cosmetic composition according to the invention is characterized in that the wax is a mineral wax or a synthetic wax chosen from the group comprising: microcrystalline waxes, paraffins, ozokerite, polyethylene waxes, silicone waxes, fluorinated waxes.

By “vegetable wax” is meant a composition comprising at least one partially or completely hydrogenated oil. These vegetable waxes may comprise esters of fatty acids and fatty alcohols, and may further comprise fatty acids, free fatty alcohols, long chain linear alkanes and/or unsaponifiables.

Preferably, within the meaning of the present invention, the waxes used are vegetable waxes.

In one embodiment, the cosmetic composition according to the invention is characterized in that the wax is a vegetable or animal wax chosen from the group comprising: lanolin wax, rice bran wax, carnauba wax, candelilla wax, berry wax, beeswax, sunflower wax, mimosa wax, jojoba wax, castor wax, olive wax.

In one embodiment, the cosmetic composition according to the invention is characterized in that the mass percentage of the wax is at least 2% relative to the total mass of the cosmetic composition.

In one embodiment, the cosmetic composition according to the invention is characterized in that the mass percentage of the wax is at least 5% relative to the total mass of the cosmetic composition.

In one embodiment, the cosmetic composition according to the invention is characterized in that the mass percentage of the wax is between 2% and 20% relative to the total mass of the cosmetic composition.

In one embodiment, the cosmetic composition according to the invention is characterized in that the mass percentage of the wax is between 5% and 15% relative to the total mass of the cosmetic composition.

In one embodiment, the cosmetic composition according to the invention is characterized in that the mass percentage of the wax is between 2% and 10% relative to the total mass of the cosmetic composition.

In one embodiment, the cosmetic composition according to the invention is characterized in that the mass percentage of the wax is between 5% and 10% relative to the total mass of the cosmetic composition.

In one embodiment, the cosmetic composition according to the invention is characterized in that it comprises a mixture according to the invention and at least one antioxidant.

In one embodiment, the cosmetic composition according to the invention is characterized in that the antioxidant is chosen from the group comprising: phenolic antioxidants of the BHA, BHT, DBPC, gallate type, so-called natural antioxidants such as dl-alpha-tocopherol or its derivatives, plant extracts, dibasic lipopeptides such as lysine or arginine, and mixtures thereof.

In one embodiment, the cosmetic composition according to the invention is characterized in that the mass percentage of the antioxidant is at least 0.01% relative to the total mass of the cosmetic composition.

In one embodiment, the cosmetic composition according to the invention is characterized in that the mass percentage of the antioxidant is at least 0.1% relative to the total mass of the cosmetic composition.

In one embodiment, the cosmetic composition according to the invention is characterized in that the mass percentage of the antioxidant is between 0.01% and 2% relative to the total mass of the cosmetic composition.

In one embodiment, the cosmetic composition according to the invention is characterized in that the mass percentage of the antioxidant is between 0.01% and 1% relative to the total mass of the cosmetic composition.

In one embodiment, the cosmetic composition according to the invention is characterized in that the mass percentage of the antioxidant is between 0.1% and 1% relative to the total mass of the cosmetic composition.

In one embodiment, the cosmetic composition according to the invention is characterized in that the mass percentage of the antioxidant is between 0.01% and 0.1% relative to the total mass of the cosmetic composition.

In one embodiment, the cosmetic composition according to the invention is characterized in that it comprises a mixture according to the invention and at least one pigmentary part.

This pigmentary part allows to obtain a coloring of the cosmetic composition. The pigments used are of synthetic, mineral or vegetable origin. Their dosage is also subtle.

In one embodiment, the cosmetic composition according to the invention is characterized in that the pigmentary part is chosen from the group comprising: water-soluble dyes, fat-soluble dyes, pigments, mother-of-pearl, glitter and mixtures thereof.

In one embodiment, the cosmetic composition according to the invention is characterized in that the pigmentary part is chosen from the group comprising: organic pigments such as fixed pigments or precipitated pigments, mineral pigments such as titanium dioxide or iron oxides, sodium aluminosilicate thiosulfates, chromium oxides, bismuth oxychloride, copper phthalocyanine, metal-free phthalocyanine, chlorinated copper phthalocyanine.

Thus, the titanium oxide rate determines the coverage and opacity of the film.

The use of lakes or precipitated pigments determines the color and chrominance.

For illustration purposes, other types of pigments can be used such as titanium mica, borosilicates and their derivatives, mica, silica.

Mineral pigments are used to nuance the hues while compounds such as bismuth oxychloride can be used to impart particular effects (metallic, shimmering effects, etc.).

In one embodiment, the cosmetic composition according to the invention is characterized in that the pigmentary part comprises a synthetic dye chosen from the group comprising: CI100006, CI12010, CI12085, CI12120, CI12370, CI12420, CI 12490, CI14270, CI14700, CI14720, CI14815, CI15525, CI15580, CI15620, CI15630, CI15850, CI15865, CI15880, CI15980, CI15985, CI1035, CI16255, CI16290, CI17200, CI18050, CI45100, CI45220, CI45380, CI45430.

In one embodiment, the cosmetic composition according to the invention is characterized in that the pigmentary part comprises a synthetic dye chosen from the group comprising: CI77499, CI77267, CI77268, CI77499 CI77266, CI50420, CI27755, CI28440, CI20470 and their mixtures.

In one embodiment, the cosmetic composition according to the invention is characterized in that the pigmentary part comprises a dye of plant origin chosen from fruit and/or plant extracts containing dyes.

In one embodiment, the cosmetic composition according to the invention is characterized in that the mass percentage of the pigmentary part is at least 0.01% relative to the total mass of the cosmetic composition.

In one embodiment, the cosmetic composition according to the invention is characterized in that the mass percentage of the pigmentary part is at least 1% relative to the total mass of the cosmetic composition.

In one embodiment, the cosmetic composition according to the invention is characterized in that the mass percentage of the pigmentary part is at least 5% relative to the total mass of the cosmetic composition.

In one embodiment, the cosmetic composition according to the invention is characterized in that the mass percentage of the pigmentary part is between 0.01% and 20% relative to the total mass of the cosmetic composition.

In one embodiment, the cosmetic composition according to the invention is characterized in that the mass percentage of the pigmentary part is between 1% and 20% relative to the total mass of the cosmetic composition.

In one embodiment, the cosmetic composition according to the invention is characterized in that the mass percentage of the pigmentary part is between 5% and 20% relative to the total mass of the cosmetic composition.

In one embodiment, the cosmetic composition according to the invention is characterized in that the mass percentage of the pigmentary part is between 1% and 15% relative to the total mass of the cosmetic composition.

In one embodiment, the cosmetic composition according to the invention is characterized in that the mass percentage of the pigmentary part is between 5% and 15% relative to the total mass of the cosmetic composition.

In one embodiment, the cosmetic composition according to the invention is characterized in that the mass percentage of the pigmentary part is between 0.01% and 5% relative to the total mass of the cosmetic composition.

In one embodiment, the cosmetic composition according to the invention is characterized in that the mass percentage of the pigmentary part is between 5% and 10% relative to the total mass of the cosmetic composition.

In one embodiment, the cosmetic composition according to the invention is characterized in that the mass percentage of the pigmentary part is between 10% and 15% relative to the total mass of the cosmetic composition.

In one embodiment, the cosmetic composition according to the invention is characterized in that it comprises a mixture according to the invention and at least one surfactant.

For the purposes of the present invention, surfactant is understood to mean a compound which modifies the surface tension between two surfaces, phases. Generally speaking, these compounds are amphiphilic molecules and make it possible to solubilize two immiscible phases of a composition.

In one embodiment, the cosmetic composition according to the invention is characterized in that the at least one surfactant is chosen from the group comprising nonionic, anionic and amphoteric surfactants.

In one embodiment, the cosmetic composition according to the invention is characterized in that the at least one surfactant is a non-ionic surfactant.

In one embodiment, the cosmetic composition according to the invention is characterized in that the at least one surfactant is chosen from the group of nonionic surfactants consisting of: polyoxyalkylene alkyl ethers, glycerin alkyl ethers, glycerin fatty acid esters, polyglycerin fatty acid esters, sorbitan fatty acid esters, their derivatives, and their mixtures.

In one embodiment, the cosmetic composition according to the invention is characterized in that the at least one surfactant is an anionic surfactant.

In one embodiment, the cosmetic composition according to the invention is characterized in that the at least one surfactant is an anionic surfactant chosen from the group consisting of: alkyl phosphates, polyoxyalkylene alkyl ether phosphates, sulfonates, alkyl sulfates, polyaspartates, their derivatives and their mixtures.

In one embodiment, the cosmetic composition according to the invention is characterized in that the at least one surfactant is an amphoteric surfactant.

In one embodiment, the cosmetic composition according to the invention is characterized in that the mass percentage of the at least surfactant is at least 0.1% relative to the total mass of the cosmetic composition.

In one embodiment, the cosmetic composition according to the invention is characterized in that the mass percentage of the at least surfactant is at least 5% relative to the total mass of the cosmetic composition.

In one embodiment, the cosmetic composition according to the invention is characterized in that the mass percentage of the at least one surfactant is between 0.1% and 30% relative to the total mass of the cosmetic composition.

In one embodiment, the cosmetic composition according to the invention is characterized in that the mass percentage of the at least one surfactant is between 5% and 15% relative to the total mass of the cosmetic composition.

In one embodiment, the cosmetic composition according to the invention is characterized in that it comprises a mixture according to the invention and at least one aqueous phase.

In one embodiment, the cosmetic composition according to the invention is characterized in that the aqueous phase consists of water.

In one embodiment, the cosmetic composition according to the invention is characterized in that the aqueous phase comprises water and at least one water-miscible organic solvent.

In one embodiment, the cosmetic composition according to the invention is characterized in that the aqueous phase comprises at least one water-miscible organic solvent chosen from the group consisting of lower alcohols such as ethanol, isopropanol, butanol, isoamyl alcohol, glycols such as ethylene glycol, propylene glycol, 1,3-butylene glycol, ketones and short-chain C₂-C₄ aldehydes.

In one embodiment, the cosmetic composition according to the invention is characterized in that the mass percentage of the aqueous phase is between 1 and 95% relative to the total mass of the cosmetic composition.

In one embodiment, the cosmetic composition according to the invention is characterized in that the mass percentage of the aqueous phase is between 5 and 80% relative to the total mass of the cosmetic composition.

In one embodiment, the cosmetic composition according to the invention is characterized in that the mass percentage of the aqueous phase is between 5 and 60% relative to the total mass of the cosmetic composition.

In one embodiment, the cosmetic composition according to the invention is characterized in that the mass percentage of the aqueous phase is between 1 and 50% relative to the total mass of the cosmetic composition.

In one embodiment, the cosmetic composition according to the invention is characterized in that the mass percentage of the aqueous phase is between 1 and 30% relative to the total mass of the cosmetic composition.

In one embodiment, the cosmetic composition according to the invention is characterized in that the mass percentage of the aqueous phase is between 10 and 40% relative to the total mass of the cosmetic composition.

In one embodiment, the cosmetic composition according to the invention is characterized in that the mass percentage of the aqueous phase is between 1 and 20% relative to the total mass of the cosmetic composition.

In one embodiment, the cosmetic composition according to the invention is characterized in that it has a naturalness index greater than or equal to 80% carbon 14 (¹⁴C) determined according to one of the following standards ASTM D6866, EN 16640 or EN 16785-1.

In one embodiment, the cosmetic composition according to the invention is characterized in that it has a naturalness index greater than or equal to 85% carbon 14 (¹⁴C) determined according to one of the following standards ASTM D6866, EN 16640 or EN 16785-1.

In one embodiment, the cosmetic composition according to the invention is characterized in that it has a naturalness index greater than or equal to 90% carbon 14 (¹⁴C) determined according to one of the following standards ASTM D6866, EN 16640 or EN 16785-1.

In one embodiment, the cosmetic composition according to the invention is characterized in that it has a naturalness index greater than or equal to 95% carbon 14 (¹⁴C) determined according to one of the following standards ASTM D6866, EN 16640 or EN 16785-1.

In one embodiment, the cosmetic composition according to the invention is characterized in that at least 85% by mass of its ingredients is biosourced relative to the total mass of the composition.

In one embodiment, the cosmetic composition according to the invention is characterized in that at least 50% by mass of its ingredients is of natural origin relative to the total mass of the composition.

In one embodiment, the cosmetic composition according to the invention is characterized in that at least 75% by mass of its ingredients is of natural origin relative to the total mass of the composition.

In one embodiment, the cosmetic composition according to the invention is characterized in that at least 90% by mass of its ingredients is of natural origin relative to the total mass of the composition.

In one embodiment, the cosmetic composition according to the invention is characterized in that 50% by mass of its ingredients is of natural origin relative to the total mass of the composition.

In one embodiment, the cosmetic composition according to the invention is characterized in that 75% by mass of its ingredients is of natural origin relative to the total mass of the composition.

In one embodiment, the cosmetic composition according to the invention is characterized in that 90% by mass of its ingredients is of natural origin relative to the total mass of the composition.

The invention also relates to the use of the mixture according to the invention for the preparation of a cosmetic composition for lipstick, long-lasting lipstick or lip gloss.

For the purposes of the present invention, the term “long-lasting” lipstick, also known as “non-transfer” lipstick, means a lipstick that does not leave marks, is water-resistant and sometimes oil-resistant.

Similarly, “lip shine” or “lip gloss” is a makeup product that gives a shiny and/or wet look to the lips, and sometimes, to color or even glitter them. Some of these lip glosses are transparent and can be applied over a classic lipstick to add a shiny effect.

Traditionally, according to the work Conception des produits cosmétiques: la formulation, by Anne-Marie PENSE-LHERITIER published in 2014 by LAVOISIER, the composition of a lipstick is structured around a pair:
base or support
Pigmentary part

The base contains different types of ingredients selected mainly on their organoleptic properties:
solvents to facilitate application and hold;
oils to provide slip and ease of application;
waxes, to fix consistency and hardness;
substances such as pasty fats or gelling agents to adjust the smoothness and ease of use.

Obtaining a lipstick is done by finding a subtle balance between these different ingredients.

These ingredients can be supplemented with various additives providing specific properties: polymers, absorbent powders, stabilizing additives (antioxidants, preservatives, etc.), perfume and active ingredients where applicable.

In one embodiment, the cosmetic compositions for long-lasting lipstick, or lip glosses comprising the mixture according to the invention exhibit excellent stability and remarkable cosmetic qualities such as ease of application, water resistance, good hold, intense and luminous color.

The invention also relates to the use of the mixture according to the invention for the preparation of a cosmetic makeup composition for the eyes.

In one embodiment, the mixture according to the invention is used for the preparation of a cosmetic mascara or eyeliner composition.

Traditionally, according to the book Conception des produits cosmétique: la formulation, by Anne-Marie PENSE-LHERITIER published in 2014 by LAVOISIER, there are mainly two types of mascara formulations:
Continuous water-phase mascaras are also called “water-based mascara”
Continuous oil phase mascaras are also called “waterproof mascara”

In one embodiment, the mixture according to the invention is used for the preparation of cosmetic compositions of mascaras with a continuous oil phase.

Continuous oil phase mascaras are mainly based on the use of wax or continuous oil phase emulsion. The aim is to form a continuous film on the surface of the eyelash, a film that is perfectly water-resistant.

To do this, the mascara composition includes in particular an oily phase and a pigmentary part and this composition may optionally include an aqueous phase.

In one embodiment, the mascara composition comprising the mixture according to the invention comprises a mass percentage of less than or equal to 10% of water relative to the total mass of the composition.

In a preferred embodiment, the mascara composition comprising the mixture according to the invention is free of an aqueous phase.

The fatty phase includes in particular, volatile solvents, oils, surfactants, waxes, gelling agents, film-forming polymers, and preservatives.

The pigment part includes water-soluble dyes, fat-soluble dyes, pigments, pearlescent agents and glitters.

Obtaining a mascara is done by finding a subtle balance between these different ingredients.

These ingredients can be supplemented with various additives providing specific properties: polymers, absorbent powders, stabilizing additives such as antioxidants, perfume and active ingredients where appropriate.

Regarding eyeliners, their composition is similar to that of the mascaras developed above.

In one embodiment, the cosmetic compositions for the eyes, such as mascara or eyeliner, comprising the mixture according to the invention have excellent stability and remarkable cosmetic qualities such as ease of application, water resistance, good hold and more intense color.

The invention also relates to the use of the mixture according to the invention for the preparation of a cosmetic composition applicable to the skin.

In one embodiment, the mixture according to the invention is particularly used for the preparation of cosmetic compositions such as a makeup primer or base, a blush, an eye shadow, a foundation or a makeup remover formulation.

Foundation is a formulation intended to unify the skin and lightly color it. It must be applied in a uniform layer but also allow blending, on the face and part of the neck.

Similarly, we understand by base, primer or makeup base a cosmetic composition that allows to blur skin imperfections and prevents makeup from settling in wrinkles, pores throughout the day. Thus, this formulation allows to perfect the skin before applying a foundation, a blush, etc.

Blush, also commonly called “blush”, is a cosmetic formulation intended to mimic or reinforce a “rosy cheek” appearance.

Similarly, eye shadow or commonly called “eye-shadow” means cosmetic formulations applicable to the eyelid in order to highlight the eyes by creating color contrasts.

By makeup remover formulation we mean a makeup remover oil but also a biphasic makeup remover formulation which allows the face to be cleansed of makeup while respecting the skin of its user.

As stated above, these makeup remover formulations can come in the form of a makeup remover oil made up of oils and various fatty substances.

They can also be presented in the form of a biphasic formulation comprising an aqueous phase and an oily phase.

Conventionally, the mixture according to the invention is used for the preparation of cosmetic compositions applicable to the skin, in particular foundation and/or base compositions which can be formulated in a fluid or solid form of powder type, loose compact or cast.

These compositions may in particular, independently of one another, be presented in an anhydrous form or in the form of an emulsion.

These emulsions can come in different forms, namely in the form of an oil-in-water emulsion, water-in-oil, but also under certain conditions in the form of a multiple W/O/W (water/oil/water) or even O/W/O (oil/water/oil) emulsion combining at least one aqueous phase and one fatty phase.

In one embodiment, the mixture according to the invention is used for the preparation of a cosmetic composition applicable to the skin comprising at least one aqueous phase and one fatty phase.

The aqueous phase consists mainly of water.

The fatty phase contains different types of ingredients selected mainly on their organoleptic properties:
solvents to facilitate application and hold
oils to provide slip and ease of application
waxes, to fix consistency and hardness
fatty substances such as pasty fats or gelling agents to adjust the smoothness and ease of use.

Generally, the mixture according to the invention is used for the preparation of a cosmetic composition applicable to the skin comprising a pigmentary part.

Obtaining a cosmetic composition that is applicable to the skin is achieved by finding a subtle balance between these different ingredients.

These ingredients can be supplemented with various additives providing specific properties: polymers, absorbent powders, stabilizing additives (antioxidants, preservatives, etc.), perfume and active ingredients where applicable.

In one embodiment, the cosmetic compositions applicable to the skin such as bases or primers, foundations or powders, comprising the mixture according to the invention have excellent stability and remarkable cosmetic properties such as ease of application, water resistance, good hold, more intense and luminous color.

The invention also relates to the use of the mixture according to the invention for the preparation of a cosmetic composition for the treatment of keratin fibers, in particular hair.

In one embodiment, the mixture according to the invention is used for the preparation of cosmetic compositions for the treatment of keratin fibers in the form of a solution, dispersion, oil/water emulsion, water/oil emulsion, water/oil/water emulsion, powder, talc, sponge, mousse, lacquer and gel.

In one embodiment, the mixture according to the invention is used for the preparation of cosmetic compositions for the treatment of keratin fibers such as hair toners, hair dyes, hair styling products, shampoos, hair conditioners, conditioners, conditioning shampoos, hair treatment products, hair sprays, hair masks, hair care products, hair treatment products, permanent wave fixing solutions, hair shaping products, shampoos for colored hair, hair fixatives, hair holding products, hair styling preparations, leave-in hair products, curling iron lotions, fixing mousses, hair gels, hair waxes or combinations thereof.

In one embodiment, the cosmetic compositions for the treatment of keratin fibers, in particular hair, comprising the mixture according to the invention exhibit excellent stability and remarkable cosmetic properties such as ease of application, good hold, luminous color and intense shine.

The following examples are given to illustrate the invention.

### Method for Determining Viscosity

Viscosity is determined using a Brookfield type automatic viscometer.

The type of mobile and the speed of the mobile are adapted to the measurement range.

of the measurement is the dynamic viscosity expressed in cP or mPa·s.

This value divided by the density of the product gives the kinematic viscosity expressed in cSt or mm²·s⁻¹.

### Method for Determining the Acid Index

The quantity of free acids is determined using the NF EN ISO 660 method.

### Method for Determining the Hydroxyl Index

The amount of free hydroxyls is determined using the NF ISO 4629 4629-2 method.

### Method for Determining Density

Density is measured using a Mettler Toledo DENSITO device. It is expressed in g·cm⁻³.

### Method of Measuring Refractive Index

The refractive index is measured using an automatic device of the type Model RFM330-M, Belligham and Stanley Ltd.

### EXAMPLE 1

### Process for Preparing a Mixture According to the Invention

In a 700 L reactor equipped with a heating system, stirring and a vacuum system are loaded 47.5 kg of diacid C₃₆ (Pripol 1009, Croda), 54 kg of diol C₃₆ (Pripol 2033, Croda) and 41.4 kg of squalane (Squalive, Biosynthis).

The pressure is lowered to 10 mbar while the temperature of the stirred mixture is gradually increased to 220° C.

5 kg of water are recovered by condensation during the esterification reaction.

The temperature of the reaction medium is then lowered to 100° C. and the pressure is returned to atmospheric pressure.

The resulting mixture, 138 kg, is composed of 70% polyester and 30% squalane by mass.

The mixture according to the invention has a viscosity of 384,515 cSt at 40° C., a density of 0.8699 at 40° C., an acid number of 0.34 mg KOH/g, a refractive index of 1.4775 at 20° C. and a hydroxyl number of 10 mg KOH/g.

### EXAMPLE 2

### Process for Preparing a Mixture According to the Invention

In a 700 L reactor equipped with a heating system, stirring and a vacuum system are loaded 47.5 kg of diacid C₃₆ (Pripol 2009, Croda), 54 kg of diol C₃₆ (Pripol 2033, Croda), 41.4 kg of squalene (Vegetable squalene ST, Biosynthis) and 0.1 kg of methanesulfonic acid.

The pressure is lowered to 10 mbar while the temperature of the stirred mixture is gradually increased to 160° C.

5 kg of water are recovered by condensation during the esterification reaction.

The temperature of the reaction medium is then lowered to 100° C. and the pressure is returned to atmospheric pressure.

The resulting mixture, 138 kg, is composed of 70% polyester and 30% squalene by mass.

The mixture according to the invention has a viscosity of 266,007 cSt at 40° C., a density of 0.8699 at 40° C., an acid number of 0.81 mg KOH/g, a refractive index of 1.4906 at 20° C. and a hydroxyl number of 10 mg KOH/g.

### EXAMPLE 3

### Process for Preparing a Mixture According to the Invention

In a 700 L reactor equipped with a heating system, stirring and a vacuum system are loaded 47.5 kg of diacid C₃₆ (Pripol 1009, Croda), 54 kg of diol C₃₆ (Pripol 2033, Croda), 41.4 kg of a mixture of alkanes C₁₆-C₁₈ (Vegelight 1618, Biosynthis) and 0.1 kg of tin oxalate.

The pressure is lowered to 200 mbar while the temperature of the stirred mixture is gradually increased to 170° C.

5 kg of water are recovered by condensation during the esterification reaction.

The temperature of the reaction medium is then lowered to 100° C. and the pressure is returned to atmospheric pressure.

The resulting mixture, 138 kg, is composed of 70% polyester and 30% of a mixture of C₁₆-C₁₈ alkanes by mass.

The mixture according to the invention has a viscosity of 120080 cSt at 40° C., a density of 0.8436 at 40° C., an acid number of 0.20 mg KOH/g, a refractive index of 1.4697 at 20° C. and a hydroxyl number of less than 10 mg KOH/g.

### EXAMPLE 4

### Cosmetic Hair Serum Composition Comprising a Mixture According to the Invention

A cosmetic hair serum composition is prepared from the mixture according to the invention prepared according to the process described in Example 1.

This mixture is composed of 70% by mass of polyester resulting from the reaction between the diacid C₃₆ (Pripol 1009, Croda) and the diol C₃₆ (Pripol 2033, Croda) and 30% by mass of squalane.

The detailed composition of this cosmetic hair serum formulation is given in the table below:

TABLE 1
Cosmetic composition of a hair serum

| | | | % by |
|---|---|---|---|
| Phase | Trade name | INCI | mass |
| 1 | MIXTURE ACCORDING TO THE INVENTION | | 31 |
| | VEGELIGHT I SILK | C9-12 ALKANE | 67 |
| 2 | KARILEINE OIL | BUTYROSPERMUM PARKII | 2 |
| | BIOSYNTHIS | (SHEA) BUTTER | |

| | |
|---|---|
| TOTAL | 100 |

Phase 1 ingredients are dispersed in Ultra-Turrax™.

Phases 1 and 2 are then mixed to obtain a hair serum.

The cosmetic hair serum composition comprising a mixture according to the invention has the following characteristics: a refractive index of 1.4348 at 20° C. and a viscosity of 33 cSt at 25° C.

### EXAMPLE 5

### Cosmetic Mascara Composition Comprising a Mixture According to the Invention

A cosmetic mascara composition is prepared from a mixture according to the invention prepared according to the process described in Example 2.

This mixture is composed of 70% by mass of polyester resulting from the reaction between the diacid C₃₆ (Pripol 1009, Croda) and the diol C₃₆ (Pripol 2033, Croda) and 30% by mass of squalene.

The detailed composition of this cosmetic hair serum formulation is given in the table below:

TABLE 2

| | | | % by |
|---|---|---|---|
| Phase | Trade name | INCI | mass |
| 1 | VEGELIGHT I SILK | C9-12 ALKANE | 25.3 |
| | VEGELIGHT BENTONE | C9-12 ALKANE & STEARALKONIUM | 10 |
| | SILK SB | BENTONITE & | |
| | | TRIETHYL CITRATE | |
| | TMS 803 | TRIMETHYLSILOXYSILICATE | 1 |
| | MI BLACK | ALUMINUM DIMYRISTATE, CI 77499 | 14 |
| | MASSOCARE PEG30 | PEG-30 DIPOLYHYDROXYSTEARATE | 1 |
| | PHS | | |
| | MIXTURE ACCORDING TO THE INVENTION | | 5 |
| 2 | ANTARON V216 | PVP/HEXADECENE COPOLYMER | 3 |
| | (GANEX) | | |
| | KAHLRESIN 6723 | SHOREA ROBUSTA RESIN & | 1.5 |
| | | OCTYLODECANOL | |
| | MYRITOL 318 | CAPRYLIC/CAPRIC TRIGLYCERIDE | 1.8 |
| | CEGESOFT HF 52 | HYDROGENATED VEGETABLE OIL | 10 |
| | BEESWAX WHITE SP | BEESWAX | 3 |
| | 422P | | |
| | CARNAUBA WAX SP | COPERNICIA CERIFERA (CARNAUBA) | 2.5 |
| | 63P | WAX | |
| | CRODABOND CSA LQ | HYDROGENATED CASTOR OIL/SEBACIC | 3 |
| | | ACID COPOLYMER | |
| | PARLEAM | HYDROGENATED POLYISOBUTENE | 2 |
| 3 | PHENOXETOL ® | PHENOXYETHANOL | 0.7 |
| | DERMOSOFT OCTIOL | CAPRYLYL GLYCOL | 0.5 |
| 4 | DEIONIZED WATER | WATER | 10 |
| | POTASSIUM SORBATE | POTASSIUM SORBATE | 0.2 |
| 5 | SILICA BEAD SB 700 | SILICA | 2 |
| | MIYOSYN MATTE-ALT | MICA (AND) ISOPROPYL TITANIUM | 2 |
| | | TRIISOSTEARATE | |
| | SENSOSEL 10 | CELLULOSE | 1 |
| | ESP ECOCEL 10 | CELLULOSE | 0.5 |

| | |
|---|---|
| TOTAL | 100 |

The ingredients of phase 1 are mixed and the mixture is heated to a maximum of 40° C.

The ingredients of phase 2 are mixed and heated to 60° C.

Phase 2 is added to Phase 1.

Then, phase 3 is added to the mixture of phases 1 and 2.

Phase 4 is gradually added to the mixtures of phases 1, 2 and 3 with vigorous stirring.

Finally, the ingredients from phase 5 are added to the mixture from phases 1, 2, 3 and 4.

### EXAMPLE 6

### Process for Preparing an Ultra-Viscous Viscoplast

In a 700L reactor, equipped with a heating system, stirring and a vacuum system are loaded: 166 kg of diacid C₃₆ (Pripol 1009 from CRODA), 30 kg of 1,4 Butanediol (1,4BDO from BRENNTAG), 4 kg of diol C₃₆ (Pripol 2033 from CRODA) and 2 kg of catalyst B, which is tin oxalate.

The mixture is stirred and the pressure is gradually lowered to a pressure of 10 mbar.

The temperature is then gradually raised to 170° C.

During esterification, 12 kg of water and excess BDO are evaporated.

The temperature is then lowered to 100° C. and the pressure is returned to atmospheric pressure.

The resulting mixture, 200 kg, is composed of 100% ultra-viscous Viscoplast.

The ultra-viscous Viscoplast has a molecular weight of 142,261 g/mol as measured by Size Exclusion Chromatography (SEC), and it exhibits a viscosity of 500,000 cSt, a density at 40° C. of 0.9500, a hydroxyl number of 30 mg KOH/g, an acid number of 2.0 mg KOH/g and a refractive index at 20° C. of 1.4853.

### EXAMPLE 7

### Process for Preparing an Ultra-Viscous Viscoplast

In a 300L reactor, equipped with a heating system, stirring and a vacuum system are loaded: 166 kg of diacid C₃₆ (pripol 1009 from croda), 27 kg of 1,4 Butanediol (1,4BDO from brenntag), and 0.2 kg of acid catalyst A, which is methanesulfonic acid. The mixture is stirred and the pressure is gradually lowered to a pressure of 10 mbar. The temperature is then gradually raised to 170° C. During the esterification, 10.5 kg of water and excess BDO are evaporated. The temperature is then lowered to 100° C. and the pressure is returned to atmospheric pressure. The reaction mixture is withdrawn 182.7 kg and is composed of 100% ultra-viscous viscoplast.

This composition has the following characteristics: a viscosity of 1,600,000 cST, a density at 40° C. of 0.9500, an acid index of 1.7 mg KOH/g and a refractive index at 20° C. of 1.4853.

In comparison, a Viscoplast prepared according to a prior art protocol such as VISCOPLAST GREEN 3000, having a viscosity of 3000 cSt, and has an average molecular mass of 20,602 g/mol measured by Size Exclusion Chromatography (SEC) as well as a density of 0.9500.

In conclusion, the Viscoplasts obtained by a process according to the invention exhibit both lower hydroxyl indices than those of the prior art, while exhibiting higher viscosities and molecular masses.

1-24. (canceled)

25. Mixture comprising:
a) at least one polyester resulting from a reaction between at least one dicarboxylic acid dimer and at least one alcohol bearing at least two hydroxyl functions, the at least one polyester having a viscosity of at least 200,000 cSt (mm²·s⁻¹) at 40° C. and a hydroxyl index of less than 30 mg KOH/g, said viscosity being determined using an automatic Brookfield type viscometer, the type of spindle and the speed of the spindle being adapted to the measurement range, and said hydroxyl index being measured according to the NF ISO 4629-2 method,
b) at least one biosourced hydrocarbon compound, namely a compound formed essentially, or even consisting of, carbon and hydrogen atoms, and optionally oxygen and nitrogen atoms, wherein the organic carbon present in said compound is 100% of plant origin (¹⁴C) by radiocarbon analysis according to one of the following standards ASTM D6866, EN 16640 or EN 16785-1,
and said mixture being wherein the mass percentage of the at least one polyester is at least 50% relative to the total mass of the mixture and the mass percentage of the at least one biosourced hydrocarbon compound is at least 5% relative to the total mass of the mixture.

26. Mixture according to claim 25, wherein the at least one polyester is derived from a reaction between at least one dicarboxylic acid dimer of plant origin and at least one alcohol carrying at least two hydroxyl functions of plant origin.

27. Mixture according to claim 25, wherein the at least one dicarboxylic acid dimer is chosen from the group consisting of hydrogenated oleic acid dimers, hydrogenated linoleic and/or linolenic acid dimers, alone or as a mixture.

28. Mixture according to claim 27, wherein the at least one dicarboxylic acid dimer is the compound with CAS number 68783-41-5.

29. Mixture according to claim 27, wherein the at least one alcohol carrying two hydroxyl functions is chosen from the group consisting of (9Z,12Z)-18-[(6Z,9Z)-18-hydroxyoctadeca-6,9-dienoxy]octadeca-9,12-dien-1-ol (CAS 147853-32-5), 1,4-butanediol, 1,3-propanediol, 1,5-pentanediol and mixtures thereof.

30. Mixture according to claim 25 wherein the at least biosourced hydrocarbon compound is a non-volatile compound.

31. Mixture according to claim 25 wherein the at least one biosourced hydrocarbon compound is of plant origin.

32. Mixture according to claim 25 wherein the at least one biosourced hydrocarbon compound is chosen from the group consisting of linear or branched, saturated or unsaturated alkanes containing at least 12 carbon atoms, alone or as a mixture.

33. Mixture according to claim 30, wherein the at least one biosourced hydrocarbon compound is chosen from the group consisting of squalane, squalene and mixtures of C16-C18 alkanes.

34. Mixture according to claim 25, wherein it has a percentage greater than or equal to 85% of carbon 14 (14C) determined according to one of the following standards ASTM D6866, EN 16640 or EN 16785-1.

35. Mixture according to claim 25, wherein the mass percentage of the at least one polyester is between 50 and 95% relative to the total mass of the mixture and the mass percentage of the at least one biosourced hydrocarbon compound is between 5 and 50% relative to the total mass of the mixture.

36. Mixture according to claim 25, wherein it comprises at least 50% by mass of the at least one polyester resulting from a reaction between at least one dicarboxylic acid dimer and at least one alcohol carrying at least two hydroxyl functions and at least 10% by mass of at least one biosourced hydrocarbon compound.

37. Mixture according to claim 25, wherein it contains from 50 to 90% by mass of the at least one polyester resulting from a reaction between at least one dicarboxylic acid dimer and at least one alcohol carrying at least two hydroxyl functions and 10 to 50% by mass of at least one biosourced hydrocarbon compound.

38. Process for obtaining the mixture according to claim 25, wherein it comprises the following steps:
a) at least one carboxylic acid dimer is available,
b) at least one alcohol carrying at least two hydroxyl functions is available,
c) at least one bio-sourced hydrocarbon compound is available,
d) the compounds from steps a), b) and c) are mixed,
e) the pressure of the mixture is lowered to a pressure between 900 and 10 mbar,
f) the mixture is gradually heated to a target temperature between 150° C. and 250° C.,
g) the water formed during the condensation reaction is continuously removed,
h) a mixture is obtained comprising a polyester and the bio-sourced hydrocarbon compound,
wherein the compounds of steps a), b) and c) can be mixed in any order during step d).

39. Process for obtaining the mixture according to claim 38, wherein at least one catalyst is added to the mixture obtained during step d).

40. Process according to claim 38, wherein the polyester obtained in step h) has a viscosity of at least 1,000,000 cSt at 40° C. and a hydroxyl number of less than 15 mg KOH/g.

41. Process according to claim 38, wherein the dicarboxylic acid dimer is chosen from the group consisting of hydrogenated oleic acid dimers, hydrogenated linoleic and/or linolenic acid dimers, and the at least one alcohol bearing two hydroxyl functions is chosen from the group consisting of (9Z,12Z)-18-[(6Z,9Z)-18-hydroxyoctadeca-6,9-dienoxy]octadeca-9,12-dien-1-ol (CAS 147853-32-5), 1,4-butanediol, 1,3-propanediol, 1,5-pentanediol and mixtures thereof.

42. Cosmetic composition comprising a mixture according to claim 25 and at least one compound chosen from non-volatile oils, volatile oils, glossy oils, pasty fatty substances, gelling agents, film-forming polymers, waxes, antioxidants, pigments, dyes, surfactants, an aqueous phase, and mixtures thereof.

43. Cosmetic composition according to claim 42, wherein the mass percentage content of the mixture is greater than or equal to 5% relative to the total mass of the composition.

44. Cosmetic composition according to claim 42, wherein the cosmetic composition has a percentage greater than or equal to 80% of carbon 14 (14C) determined according to one of the following standards ASTM D6866, EN 16640 or EN 16785-1.
